(19) United States
(12) Patent Application Publication
(10) Pub. No.: US 2025/0073366 A1
(43) Pub. Date: Mar. 6, 2025

(54) EXTERNAL PROTECTIVE SHEATH FOR STERILIZATION WRAP

(71) Applicant: DelStar Technologies, Inc., Alpharetta, GA (US)

(72) Inventors: Jaime Zohrer, Miami, FL (US); Chris Hakanson, Alpharetta, GA (US); Travis Meadow, Alpharetta, GA (US); Bruce Choi, Alpharetta, GA (US)

(21) Appl. No.: 18/824,631

(22) Filed: Sep. 4, 2024

Related U.S. Application Data
(60) Provisional application No. 63/580,498, filed on Sep. 5, 2023.

(51) Int. Cl. A61L 2/26; A61B 50/30

(52) U.S. Cl. CPC A61L 2/26; A61B 50/30

(57) ABSTRACT

An external sheath for a sterilization container is provided. The external sheath may have a main body comprising a net having a plurality of shaped openings along its length, a first open end and a second open end. The main body may be provided with a geometry configured for a slip fit over the wrapped sterilization container and be formed of a sterilization-grade material. The external sheath may be part of a sterilization system that also includes a sterilization container having a tray and a lid, and a sheet for wrapping the sterilization container.

### CROSS-REFERENCE TO RELATED APPLICATIONS

This application claims the benefit of U.S. Provisional Application No. 63/580,498, filed Sep. 5, 2023, the complete disclosure of which is incorporated herein by reference for all purposes.

### TECHNICAL FIELD

A sterilization accessory, in particular an enclosure for wrapped sterilization containers, and more particularly an external protective sheath for maintaining the enclosure of a sterilization wrap around a sterilization container, is described.

### BACKGROUND

There are a variety of known methods for sterilizing surgical, medical and/or dental items such as devices, instruments, and related accessories like gloves and gowns, or any other items that come in direct contact with blood or tissue, for reuse or proper disposal. These sterilization methods can include: (1) steam or autoclave sterilization (saturated steam under high pressure), (2) dry heat (oven), (3) radiation (gamma radiation or x-rays), and (4) chemical sterilants such as ethylene oxide (ETO) gas, vaporized hydrogen peroxide (plasma sterilization), and other known validated sterilization methods. For example, chlorine dioxide gas, vaporized peracetic acid, and nitrogen dioxide are among the other known validated sterilization methods involving chemical sterilants. Because sterilization is a process, not a single event, all steps must be carried out correctly for sterilization to occur.

Most sterilization techniques require that the items for sterilization be held in a sterilization-safe container. For example, sterilization trays are commonly used to sterilize and store medical and/or dental instruments or implants. A sterilization tray system usually includes two main components: (a) a container, such as a tray or basket which forms the base of the container, for securing the items; and (b) a cover for the container. These two components comprise materials suitable for, and are configured for, steam sterilization or other known validated sterilization methods. In typical sterilization tray systems, the tray and cover have vertical or near vertical walls on all sides that allow for the tray and cover to mate, forming an enclosed container. In addition, the tray and cover are usually perforated to allow sterilization and high temperature gasses and fluids to enter and exit the container. In order to withstand the high temperatures, used especially in steam-based sterilization techniques, the tray and cover are most often made of a sheet metal or rigid thermoplastic.

Another essential component of most sterilization tray systems is the sterilization wrap. This wrap is used to enclose the sterilization container prior to sterilization and provide a sheet barrier to contaminants post-sterilization. The wrap is intended to allow sterilization of the enclosed items and provides a consistent bacteriological barrier to maintain the sterility of the items up to the point of use. Sterilization wraps are typically square or rectangular sheets or panels formed of a sterilization-sturdy material such as polypropylene. Following some sterilization techniques, the sterilization container is wrapped with the sterilization wrap by folding the sheet around the container to form a neatly enclosed package which remains sealed until use.

Fastening mechanisms such as straps, belts or bands are known to ensure the sterilization wrap does not inadvertently open. To prevent damage such as tearing or rips, which could lead to contamination, tray liners and corner or edge protectors can be used along with the wraps. Provided herein is an external protective sheath that serves as both a fastening mechanism to ensure the sterilization wrap does not inadvertently open, as well as a protective cover to avoid inadvertent tearing, rips, holes, or cuts of the wrap.

### SUMMARY

An external sheath for a sterilization container is described herein. The external sheath may have a main body comprising a net having a plurality of shaped openings along its length, a first open end and a second open end. The main body may be provided with a geometry configured for a slip fit over the wrapped sterilization container and be formed of a sterilization-grade material.

In one embodiment, the shaped openings can comprise diamond shaped openings, while the main body can comprise a rectangular shaped tube.

The sterilization-grade material can comprise a polypropylene, polylactic acid, polyethylene, a polybutylene adipate terephthalate, or a blend thereof. In one embodiment, the net comprises a monofilament and extruded, non-woven material. If so desired, the net may be partially or fully biodegradable.

In one embodiment, the external sheath may be configured for multiple cycles of sterilization, while in an alternative embodiment, the external sheath may be configured for single use.

The external sheath may be configured for use with different sterilization techniques, such as steam sterilization, dry heat sterilization, gamma radiation, x-rays, or chemical sterilization. Chemical sterilization techniques suitable for use with the external sheath may include those involving ethylene oxide gas, vaporized hydrogen peroxide, chlorine dioxide gas, vaporized peracetic acid, or nitrogen dioxide.

In some embodiments, the net may have a thickness in the range of about 0.050 to about 0.100 inches (or about 1.3 mm to about 2.5 mm), while in other embodiments, the net may have a thickness in the range of about 0.065 to about 0.085 inches (or about 1.7 mm to about 2.2 mm).

In some embodiments, the net may have a knot count in the range of about 40 to about 120 in the transverse direction, while in other embodiments, the net may have a knot count in the range of about 54 to about 100 in the transverse direction. In some embodiments, the net comprises strands, and the strands per inch of the net may range from about 3 to about 10 in the transverse direction (TD), and about 1 to about 9 in the machine direction (MD).

In some embodiments, the diamond shaped openings may have a length in the range of about 10 to 25 mm and a width in the range of about 5 to 15 mm, while in other embodiments, the diamond shaped openings may have a length of about 20 to about 25 mm, and a width of about 10 to about 15 mm.

A sterilization system is also described herein. The system may comprise a sterilization container having a tray and a lid, a sheet for wrapping the sterilization container, and an external sheath for fitting over the sheet wrapped sterilization container. The external sheath may include all of the features mentioned above.

It is to be understood that both the foregoing general description and the following detailed description are exemplary and explanatory only and are not restrictive of the disclosure. Additional features of the disclosure will be set forth in part in the description which follows or may be learned by practice of the disclosure.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIG. 1A illustrates a perspective view of an exemplary embodiment of an external protective sheath of the present disclosure.

FIG. 1B illustrates a cross-sectional side view of the external protective sheath of FIG. 1A.

FIG. 1C illustrates a top-down view of the external protective sheath of FIG. 1A.

FIG. 1D illustrates an enlarged view of the external protective sheath of FIG. 1C.

FIG. 2 illustrates an exemplary embodiment of a sterilization tray loaded with items for sterilization and a sterilization wrap.

FIG. 3 illustrates the external protective sheath of FIG. 1A over a wrapped sterilization container.

### DETAILED DESCRIPTION OF THE EMBODIMENTS

This description and the accompanying drawings illustrate exemplary embodiments and should not be taken as limiting, with the claims defining the scope of the present disclosure, including equivalents. Various mechanical, compositional, structural, and operational changes may be made without departing from the scope of this description and the claims, including equivalents. In some instances, well-known structures and techniques have not been shown or described in detail so as not to obscure the disclosure. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

For purposes of illustration, the embodiments are described with reference to applications for surgical, medical and/or dental instruments, implants or accessories such as gloves, gowns, masks, or the like. However, the embodiments may be implemented in any application requiring treatment of devices or items, especially for certain heat, steam, radiation, or chemical treatments.

In general, the external protective sheath relates to a component for use with sterilization systems that typically include some form of a closed lid container having a tray or basket for holding the items being sterilized, and a wrap for the container. As previously discussed, the wrap is applied prior to sterilization. The wrap is intended to allow sterilization of the enclosed items, and after the sterilization, provides a consistent bacteriological barrier to maintain the sterility of the items up to the point of use.

Once sterilized, the wrapped containers or tray systems are often stacked on top of one another and kept on shelves or in bins for storage until they are used. This is especially true in high volume use facilities such as laboratories, research centers, hospitals, or clinics. The stacking of the wrapped containers can create a potential for the inadvertent tearing, ripping or opening up of the sterilization wrap. Further, in some complex surgical, medical or dental procedures, multiple instruments or tools may be required. The numerous instruments or tools increases the overall weight of the container, with some containers weighing up to about 25 pounds, and as the wrapped container is handled during the sterilization and storage process, the corners of these heavy metal containers can cause damage such as tears, holes or cuts to its outer wrap or to the wraps of other containers nearby. To prevent these issues, a protective sheath can be provided which would ensure that the sterilization wrap remain intact, as well as provide an added layer of protection from nicks, rips or tears to the wrap.

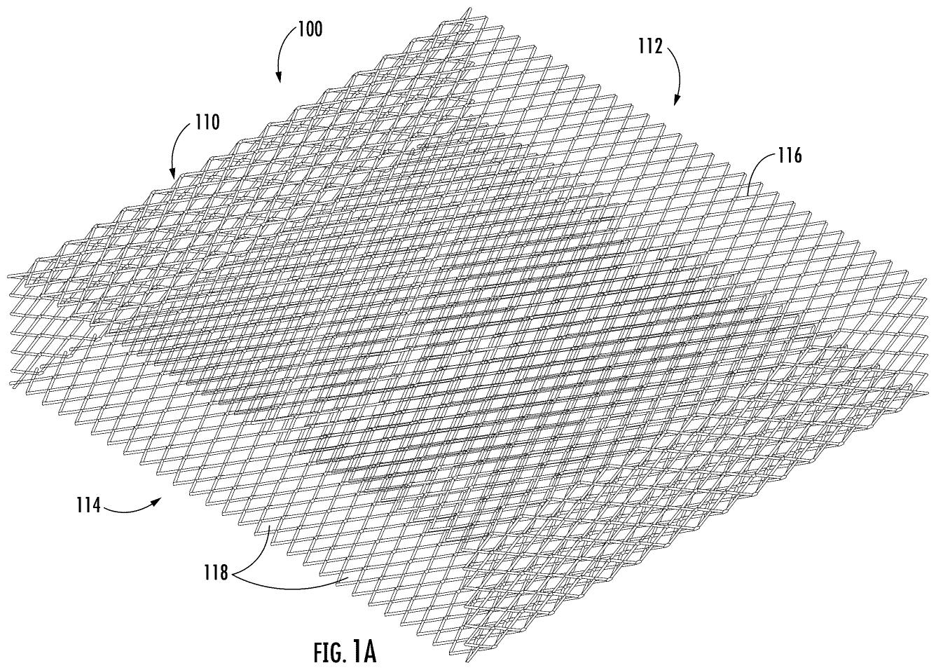

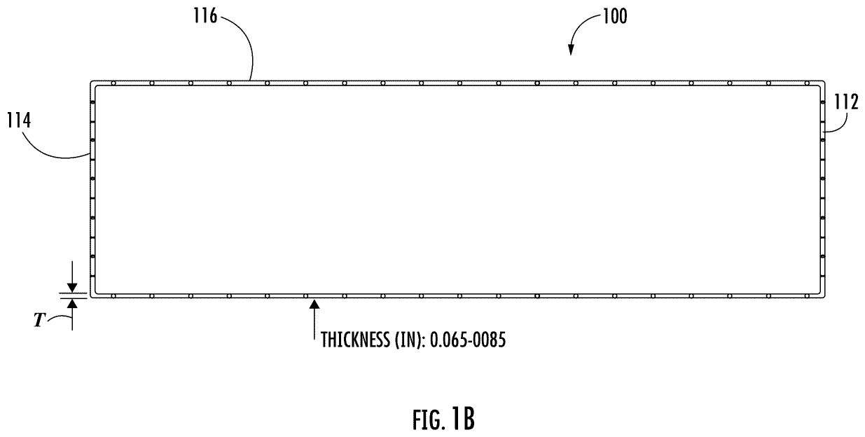

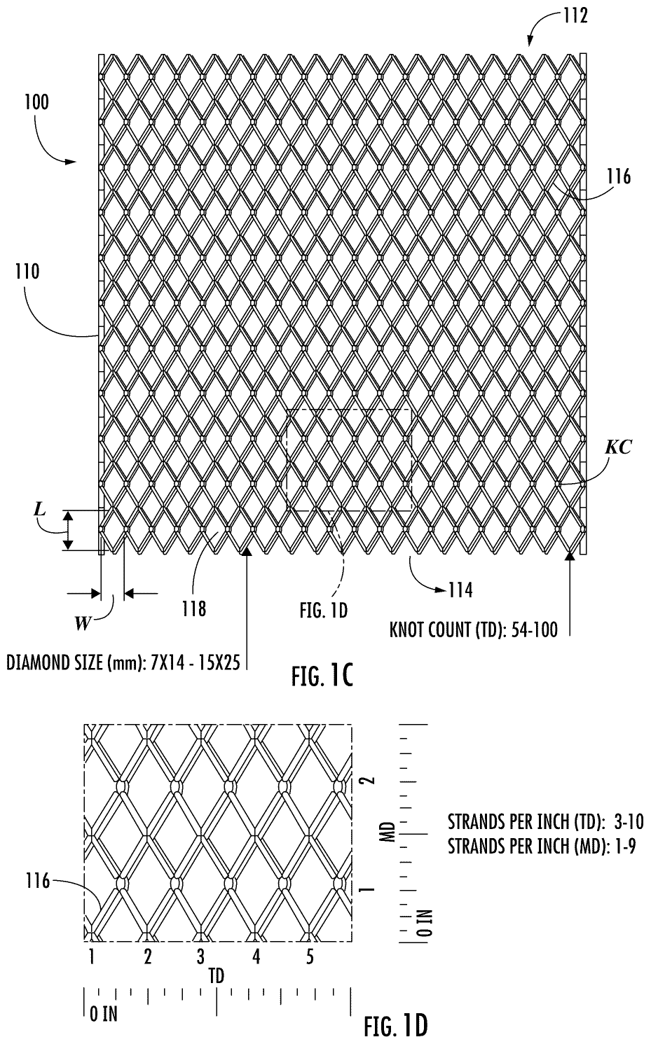

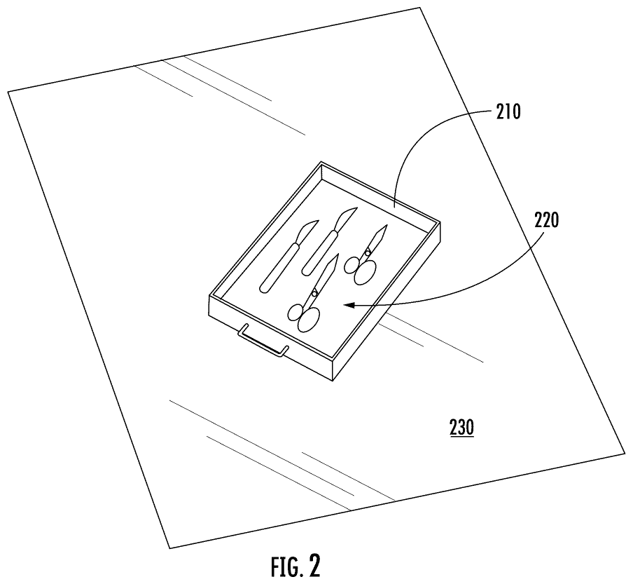

Turning now to FIG. 1A, an exemplary embodiment of an external protective sheath 100 is shown. The protective sheath 100 may have a main body 110, a first open end 112, and a second open end 114. The main body 110 may be a net formed of a plurality of strands 116 that connect at discrete points or knots (KC) along their length to form diamond shaped openings 118, as shown. As illustrated, the main body or net 110 may have a pre-formed rectangular shape that allows the external protective sheath 100 to slip over the wrapped sterilization container, as will be described in more detail below.

In some embodiments, the net 110 may have a thickness in the range of about 0.050 to about 0.100 inches (or about 1.3 mm to about 2.5 mm). As shown in FIG. 1B, in one exemplary embodiment, the net 110 may have a thickness T in the range of about 0.065 to about 0.085 inches (or about 1.7 mm to about 2.2 mm), for example.

In some embodiments, the net may have a knot count KC in the range of about 40 to about 120 in the cross, or transverse, direction (TD). For example, as shown in FIG. 1C, in one exemplary embodiment, the net 110 may have a knot count in the range of about 54 to about 100 in the transverse direction. As further shown in the enlarged view of FIG. 1D, the strands per inch may range from about 3 to about 10 in the transverse direction (TD), and about 1 to about 9 in the machine direction (MD), with the understanding that the strand count may vary by +/−3 or 4.

In some embodiments, the net 110 may comprise a plurality of diamond-shaped openings 118. The diamond-shaped openings 118 may have a size in the range of about 5×20 to 20×30 mm. In some embodiments, the diamond shaped openings 118 can have a length in the range of about 10 to 25 mm and a width in the range of about 5 to 15 mm. In some embodiments, the diamond shaped openings 118 may have a length of about 20 to about 25 mm, and a width of about 10 to about 15 mm. For example, as shown in FIG. 1C, in one exemplary embodiment, the diamond-shaped opening may have a size of between 7×14 mm to 15×25 mm, such as a length L of about 22.25 mm and a width W of about 12.60 mm. Of course, it is understood that the openings 118 are not limited to a diamond shape, and could easily work with other shapes, including but not limited to, squares, rhombuses, hexagons, as well as ovals and circles.

This net 110 may comprise a sterilization-safe, industrial-grade material, such as a polypropylene (PP) or other suitable anti-oxidant grade polymer or resin, including but not limited to polylactic acid (PLA), polyethylene (PE), or polybutylene adipate terephthalate (PBAT). In some embodiments, the net 110 may be capable of withstanding high temperature and pressures of steam sterilization. For instance, the net 110 may be designed to withstand temperatures of 270 degrees Fahrenheit or more for at least 60 consecutive minutes without compromising the integrity of the net 110. Accordingly, in the embodiments, the net 110 may have a high heat deflection value, i.e., little deformation and dimensional change due to the extended heating for a high number of cycles.

Of course, it is understood that the net 110 may be formed of materials that can withstand any form of sterilization technique, including blends of different materials. In one exemplary embodiment, the net may be formed from a monofilament and extruded, non-woven material. In some embodiments, the material may be a biodegradable material. Accordingly, the external protective sheath 100 may be provided for multiple uses or single/disposable use.

The external protective sheath 100 may be suited for a variety of different sterilization methods. These sterilization methods can include: (1) steam or autoclave sterilization (saturated steam under high pressure), (2) dry heat (oven), (3) radiation (gamma radiation or x-rays), and (4) chemical sterilants such as ethylene oxide (ETO) gas, vaporized hydrogen peroxide (plasma sterilization), and other known validated sterilization methods. For example, the external protective sheath 100 may be used with other known validated sterilization methods involving chemical sterilants like chlorine dioxide gas, vaporized peracetic acid, and nitrogen dioxide.

In an exemplary method of using the external protective sheath 100, a typical sterilization tray 210 may be loaded with items for sterilization, such as for example, surgical instruments 220, as shown in FIG. 2. Although not shown, it is well understood that a lid may be provided to cover up the sterilization tray 220 to form a fully sealed sterilization container. This sterilization container may be enclosed in a sterilization wrap 230. The sterilization wrap 230 can be provided as a typical square or rectangular sheet or panel formed of a sterilization-sturdy material such as polypropylene. The wrap 230 can be folded around the sterilization container in any conventional folding pattern to form a sheet barrier for the sterilization container.

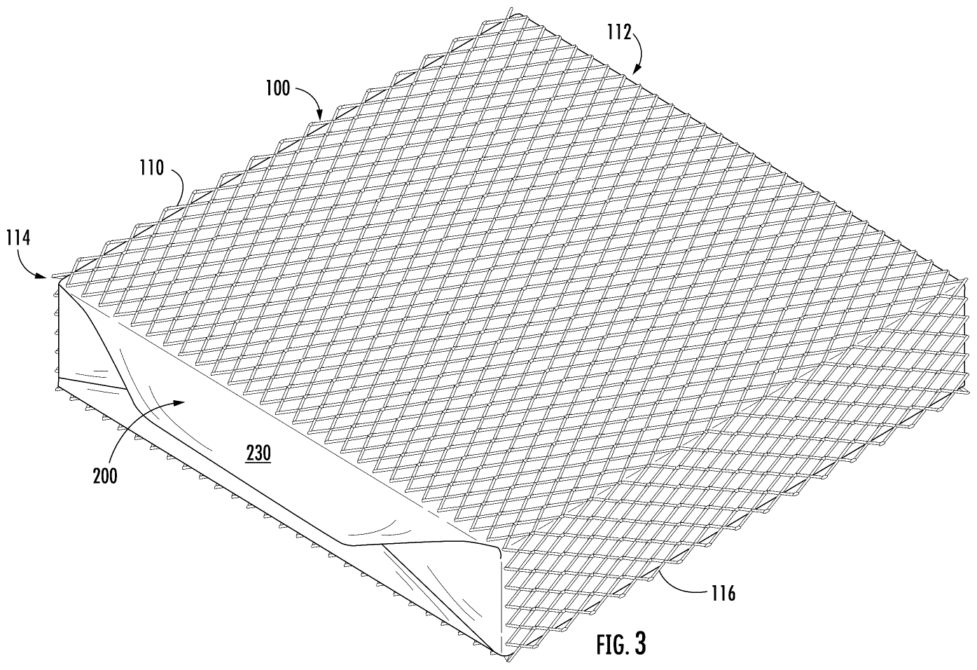

Following this step, the external protective sheath 100 may be slipped over the wrapped sterilization container 200, as shown in FIG. 3. The external protective sheath 100 should have a configuration that closely matches the geometry (size and shape) of the wrapped sterilization container 200, with sufficient clearance or space to enable the user to easily slide the external protective sheath 100 over and along the length of the wrapped sterilization container 200 without snagging or tugging on the sheath 100. However, the external protective sheath 100 should not fit so loosely as to cause overhang or bulges from extra material around the wrapped sterilization container 200 in storage, which may get caught in the handling process. For this reason, the external protective sheath 100 may be provided with a predetermined size and shape, such as the rectangular shaped tube shown in FIGS. 1A and 1B, to closely match the size and shape of the wrapped sterilization container 200. Of course, it is understood that the preformed shape of the external protective sheath 100 may include other geometries, such as a square tube. In this way, the external protective sheath 100 may act as a slipcover or slipcase, having a close fit once placed around the wrapped sterilization container 200, such as illustrated in FIG. 3.

Once properly fitted onto the wrapped sterilization container, the external protective sheath 100 may be effective at keeping the sterilization wrap 230 from inadvertently opening. Alternatively or additionally, the external protective sheath 100 may be effective in protecting the sterilization wrap 230 from tears, rips, holes, or cuts. The external protective sheath 100 may also prevent skidding of the wrapped sterilization container 200 during handling and storage. These benefits are provided without additional and unwanted bulk in thickness or weight that would negatively affect handling of the wrapped sterilization container, since the external protective sheath 100 has a thin, or low profile, and is relatively lightweight.

As previously discussed, the external protective sheath 100 may be provided for multiple uses, or as a disposable, one-time use. Further, the external protective sheath 100 may be configured to be partially or fully biodegradable after disposal.

Other embodiments will be apparent to those skilled in the art from consideration of the specification and practice of the embodiment disclosed herein. It is intended that the specification and examples be considered as exemplary only, with a true scope and spirit of the embodiment being indicated by the following claims.

For example, according to one aspect, in a 1^{st} embodiment, an external sheath for a sterilization container is provided. The external sheath may comprise a main body comprising a net having a plurality of shaped openings along its length, a first open end and a second open end. The main body may have a geometry configured for a slip fit over the wrapped sterilization container and be formed of a sterilization-grade material.

A 2^{nd} embodiment is the 1st embodiment of the external sheath, wherein the shaped openings comprise diamond shaped openings.

A 3^{rd} embodiment is any combination of the first 2 embodiments, wherein the main body comprises a rectangular shaped tube.

A 4^{th} embodiment is any combination of the first 3 embodiments, wherein the sterilization-grade material comprises a polypropylene, polylactic acid, polyethylene, a polybutylene adipate terephthalate, or a blend thereof.

A 5^{th} embodiment is any combination of the first 4 embodiments, wherein the net comprises a monofilament and extruded, non-woven material.

A 6^{th} embodiment is any combination of the first 5 embodiments, wherein the sterilization-grade material is partially or fully biodegradable.

A 7^{th} embodiment is any combination of the first 6 embodiments, wherein the external sheath is configured for multiple cycles of sterilization.

An 8^{th} embodiment is any combination of the first 7 embodiments, wherein the external sheath is configured for single use.

A 9^{th} embodiment is any combination of the first 8 embodiments, wherein the external sheath is configured for steam sterilization, dry heat sterilization, gamma radiation, x-rays, or chemical sterilization.

A 10^{th} embodiment is any combination of the first 9 embodiments, wherein the chemical sterilization includes ethylene oxide gas, vaporized hydrogen peroxide, chlorine dioxide gas, vaporized peracetic acid, or nitrogen dioxide.

An 11^{th} embodiment is any combination of the first 10 embodiments, wherein the net has a thickness in the range of about 0.050 to about 0.100 inches.

A 12^{th} embodiment is any combination of the first 11 embodiments, wherein the net has a thickness in the range of about 0.065 to about 0.085 inches.

A 13^{th} embodiment is any combination of the first 12 embodiments, wherein the net has a knot count in the range of about 40 to about 120 in the transverse direction.

A 14^{th} embodiment is any combination of the first 13 embodiments, wherein the net has a knot count in the range of about 54 to about 100 in the transverse direction.

A 15^{th} embodiment is any combination of the first 14 embodiments, wherein the net comprises strands, and the strands per inch of the net is in the range of about 3 to about 10 in the transverse direction (TD), and about 1 to about 9 in the machine direction (MD).

A 16^{th} embodiment is any combination of the first 15 embodiments, wherein the diamond shaped openings have a length in the range of about 10 to 25 mm and a width in the range of about 5 to 15 mm.

A 17^{th} embodiment is any combination of the first 16 embodiments, wherein the diamond shaped openings having a length of about 20 to about 25 mm, and a width of about 10 to about 15 mm.

According to a second aspect, in a 1st embodiment, a sterilization system is provided. The sterilization system may comprise a sterilization container having a tray and a lid, a sheet for wrapping the sterilization container, and an external sheath for fitting over the sheet wrapped sterilization container.

A 2^{nd} embodiment is the 1st embodiment of the sterilization system, wherein the external sheath comprises a main body comprising a net having a plurality of shaped openings along its length, a first open end and a second open end, the main body having a geometry configured for a slip fit over the sheet wrapped sterilization container and being formed of a sterilization-grade material.

A 3^{rd} embodiment is any combination of the first 2 embodiments, wherein the shaped openings comprise diamond shaped openings.

A 4^{th} embodiment is any combination of the first 3 embodiments, wherein the main body comprises a rectangular shaped tube.

A 5^{th} embodiment is any combination of the first 4 embodiments, wherein the sterilization-grade material comprises a polypropylene, polylactic acid, polyethylene, a polybutylene adipate terephthalate, or a blend thereof.

A 6^{th} embodiment is any combination of the first 5 embodiments, wherein the net comprises a monofilament and extruded, non-woven material.

A 7^{th} embodiment is any combination of the first 6 embodiments, wherein the sterilization-grade material is partially or fully biodegradable.

An 8^{th} embodiment is any combination of the first 7 embodiments, wherein the sheath is configured for multiple cycles of sterilization.

A 9^{th} embodiment is any combination of the first 8 embodiments, wherein the external sheath is configured for single use.

A 10^{th} embodiment is any combination of the first 9 embodiments, wherein the sterilization system is configured for steam sterilization, dry heat sterilization, gamma radiation, x-rays, or chemical sterilization.

An 11^{th} embodiment is any combination of the first 10 embodiments, wherein the chemical sterilization includes ethylene oxide gas, vaporized hydrogen peroxide, chlorine dioxide gas, vaporized peracetic acid, or nitrogen dioxide.

A 12^{th} embodiment is any combination of the first 11 embodiments, wherein the net has a thickness in the range of about 0.050 to about 0.100 inches.

A 13^{th} embodiment is any combination of the first 12 embodiments, wherein the net has a thickness in the range of about 0.065 to about 0.085 inches.

A 14^{th} embodiment is any combination of the first 13 embodiments, wherein the net has a knot count in the range of about 40 to about 120 in the transverse direction.

A 15^{th} embodiment is any combination of the first 14 embodiments, wherein the net has a knot count in the range of about 54 to about 100 in the transverse direction.

A 16^{th} embodiment is any combination of the first 15 embodiments, wherein the net comprises strands, and the strands per inch of the net is in the range of about 3 to about 10 in the transverse direction (TD), and about 1 to about 9 in the machine direction (MD).

A 17^{th} embodiment is any combination of the first 16 embodiments, wherein the diamond shaped openings have a length in the range of about 10 to 25 mm and a width in the range of about 5 to 15 mm.

An 18^{th} embodiment is any combination of the first 17 embodiments, wherein the diamond shaped openings having a length of about 20 to about 25 mm, and a width of about 10 to about 15 mm.

What is claimed is:

1. An external sheath for a sterilization container, comprising:
a main body comprising a net having a plurality of shaped openings along its length, a first open end and a second open end, the main body having a geometry configured for a slip fit over the wrapped sterilization container and being formed of a sterilization-grade material.

2. The external sheath of claim 1, wherein the shaped openings comprise diamond shaped openings.

3. The external sheath of claim 1, wherein the main body comprises a rectangular shaped tube.

4. The external sheath of claim 1, wherein the sterilization-grade material comprises a polypropylene, polylactic acid, polyethylene, a polybutylene adipate terephthalate, or a blend thereof.

5. The external sheath of claim 1, wherein the net comprises a monofilament and extruded, non-woven material.

6. The external sheath of claim 1, wherein the sterilization-grade material is partially or fully biodegradable.

7. The external sheath of claim 1, further being configured for multiple cycles of sterilization.

8. The external sheath of claim 1, further being configured for single use.

9. The external sheath of claim 1, further being configured for steam sterilization, dry heat sterilization, gamma radiation, x-rays, or chemical sterilization.

10. The external sheath of claim 9, wherein the chemical sterilization includes ethylene oxide gas, vaporized hydrogen peroxide, chlorine dioxide gas, vaporized peracetic acid, or nitrogen dioxide.

11. The external sheath of claim 1, wherein the net has a thickness in the range of about 0.050 to about 0.100 inches.

12. The external sheath of claim 11, wherein the net has a thickness in the range of about 0.065 to about 0.085 inches.

13. The external sheath of claim 1, wherein the net has a knot count in the range of about 40 to about 120 in the transverse direction.

14. The external sheath of claim 13, wherein the net has a knot count in the range of about 54 to about 100 in the transverse direction.

15. The external sheath of claim 1, wherein the net comprises strands, and the strands per inch of the net is in the range of about 3 to about 10 in the transverse direction (TD), and about 1 to about 9 in the machine direction (MD).

16. The external sheath of claim 1, wherein the diamond shaped openings have a length in the range of about 10 to 25 mm and a width in the range of about 5 to 15 mm.

17. The external sheath of claim 16, wherein the diamond shaped openings having a length of about 20 to about 25 mm, and a width of about 10 to about 15 mm.
